(19) United States
(12) Patent Application Publication
(10) Pub. No.: US 2013/0338533 A1
(43) Pub. Date: Dec. 19, 2013

(54) APPARATUS AND METHOD FOR OBTAINING TRANSEPITHELIAL SPECIMEN

(71) Applicant: David A. Olsen, Thayne, WY (US)

(72) Inventor: David A. Olsen, Thayne, WY (US)

(73) Assignee: SHARED MEDICAL RESOURCES, LLC, Newport Beach, CA (US)

(21) Appl. No.: 14/002,673

(22) PCT Filed: Mar. 14, 2012
(86) PCT No.: PCT/US12/29116

§ 371 Date: Aug. 30, 2013

Related U.S. Application Data
(60) Provisional application No. 61/452,587, filed on Mar. 14, 2011.

(51) Int. Cl. A61B 10/02

(52) U.S. Cl. CPC A61B 10/0291
USPC 600/569

(57) ABSTRACT

A non-lacerational apparatus to collect cells in a body cavity provides a brush with bristles which have an abrading surface for collect a histological sample and method for use of the apparatus.

### CROSS-REFERENCE TO RELATED APPLICATIONS

This application claims priority under 35 U.S.C. §119(e) to U.S. Provisional Application No. 61/452,587, filed on Mar. 14, 2011; the entirety of which is incorporated by reference herein for all purposes and made a part of the present specification.

### BACKGROUND

1. Field

The disclosure relates generally to the field of tissue sampling, and more particularly, to the field of obtaining transepithelial specimens.

2. Description of the Related Art

The design of prior art cytology sampling instruments reflects their use to sweep up cells which were spontaneously exfoliated and present on the superficial epithelial surface. Since prior-art cytology brushes need only to gently remove surface material, they are designed of various soft materials which can collect the cervical mucus with minimal abrasion to the underlying epithelium. These cytology sampling instruments therefore either have soft bristles, soft flexible fimbriated or fringed ends, or even, as in the case of the cotton swab or spatula, no bristles at all.

Further examples of prior art cytological sampling tools include the Cytobrush® a device which uses soft and tapered bristles to sample shed cells from the cervical canal. U.S. Pat. No. 4,759,376, which allegedly covers the Cytobrush®, likewise describes a conical tapered soft bristle brush (a mascara brush shape) which is placed into the cervical canal and rotated for endocervical sampling. U.S. Pat. No. 4,759,376 states that the bristles “are to be relatively soft such as that of a soft toothbrush to more readily bend and avoid damaging the tissues.” By way of further example, physicians have long used the common swab, commercially known as the Q-Tip®, to perform endocervical sampling.

### SUMMARY

There is still a need for an effective transepithelial non-lacerational sampling apparatus effective for targeting tissue in the endocervical canal, and other bodily orifices where suspected lesions may be found. There is a need for effective sampling of the circumferential surface of the endocervical canal, particularly since sampling is not done under direct visualization. In addition, there is a need for simultaneous and effective histologic sampling of the transformation zone and the endocervical canal.

There is also a growing, unaddressed need for an effective transepithelial non-lacerational sampling apparatus effective for targeting tissue in the anal canal. There is a growing incidence of anal lesions arising from human papillomavirus infection among other causes, which require screening sampling similar to that of the cervix. Like the endocervical canal, the anal canal is a narrow, cylindrical passageway, and routine sampling is most effectively accomplished in the office setting without the need for dilation or direct visualization such as through anoscopy. The anal mucosa, like that of the endocervix, is relatively friable, so it is particularly important to provide a non-lacerational sampling apparatus which can sample circumferentially through multiple layers of the epithelium of the anal canal.

These and other needs are addressed using apparatuses and related methods and kits for obtaining a sample of cells in epithelial tissue of the body, specifically the endocervical canal. Methods and apparatuses for obtaining transepithelial specimens of body surfaces using a non-lacerating technique are disclosed herein. Specifically, embodiments of tools for sampling squamous epithelium from the endocervical regions as well as lesions found in the oral cavity, anal canal, colon, intestine, stomach, throat, esophagus, nasal passages, trachea, bronchi, urethra, and in similar body tissues. Aspects of the embodiment are also directed to an improved method of testing all lesions that involve the epithelium of the oral cavity, endocervix, anal, throat, esophagus, nasal passages, trachea, bronchi, urethra, and/or similar body tissues.

Apparatuses and methods are useful for obtaining transepithelial tissue samples using a non-lacerating technique in body orifices, for example, in sampling squamous epithelium from lesions found in the endocervical canal and the transformation zone, which is the region between the endocervical and ectocervical region. The apparatus is intended to replace, or at least to be used in combination with, the curette used in traditional endocervical curettage. Embodiments of the apparatus are also provided for sampling the anal canal such that the shape of the brush and stiffness of the bristles are sufficient to penetrate at least two layers of epithelial tissue and obtain a sample of cells. Methods are provided for obtaining a specimen of multiple layers of epithelium in a non-lacerative manner using an endocervical apparatus. Kits are also provided for convenient and comprehensive sampling of the cervical region during, for example, colposcopy, such that apparatuses for sampling both the ectocervical and the endocervical canal are available in a single kit.

In one embodiment, an apparatus for obtaining a sample of cells in epithelial tissue of the body comprises a brush, wherein said brush comprises bristles having sufficient stiffness to penetrate at least said two layers of said epithelial tissue. In an embodiment, the brush is a non-lacerational brush. And in an embodiment, the bristles collectively substantially form the shape of a cone. In other embodiments, other shapes are possible, for example, but not limited to, spherical, pyramidal, umbrella shaped, parabolic, or the like. In an embodiment, only a distal portion of the bristles collectively substantially form a conical shape while the remainder of the bristles collectively substantially form a cylindrical shape. In an embodiment, only a distal portion of the bristles collectively substantially form a spherical, pyramidal, umbrella shaped, or parabolic shape while the remainder of the bristles collectively substantially form a cylindrical shape. This can facilitate insertion of the brush into the endocervical canal.

In another embodiment, an apparatus for obtaining a sample of cells in epithelial tissue of the body comprises a brush wherein said brush comprises bristles having sufficient stiffness to penetrate at least said two layers of said epithelial tissue, wherein said brush is a non-lacerational brush, and wherein the bristles collectively substantially form the shape of a cylinder.

In an embodiment, an apparatus for obtaining a sample of cells in epithelial tissue of the body comprises a brush, wherein said brush comprises bristles having sufficient stiffness to penetrate at least said two layers of said epithelial tissue, wherein said brush is a non-lacerational brush, and wherein the brush is shaped so that it is capable of collecting histological tissue/cells samples from an endocervical canal. In an embodiment, the brush is configured to only collect histological tissues/cells from an endocervical canal. By collecting only histological tissues/cells from an endocervical canal, and not from any other area, for example, the cervical os or surrounding ectocervical tissue, on a single brush, it can be more accurately determined that suspicious tissues/cells are located in the endocervical canal.

The apparatus may also, in some embodiments, comprise a brush which is non-invasive upon insertion to the endocervical canal. This non-invasive brush can in some embodiments comprise a sheath for shielding the brush from penetrating tissue upon entry to the endocervical canal. In some embodiments, it is the size and/or stiffness of the bristles which determines that the brush is sufficiently non-invasive.

In some embodiments, the brush of the apparatus further comprises a twisted wire wherein bristles are held in place by the twisted wire and arranged from smallest in length at the distal end of the brush to the longest bristles at the proximal end of the brush to form a spiral substantially in the shape of a cone. In other embodiments, the brush of the apparatus further comprises a twisted wire, wherein bristles are of uniform length and held in place by the twisted wire to form a spiral substantially in the shape of a cylinder.

In some embodiments, the brush of the apparatus further comprises a rounded tip or a cone-shaped tip on the distal end of the brush. In other embodiments, the tip is spherical or elongate. The tip may in some embodiments be comprised of a substance which is dipped onto the distal end of the wire.

In some embodiments, the bristles which comprise the brush are configured to collect cells from three layers of said epithelial tissue, said three layers comprising superficial, intermediate and basal layers, said basal layer separated from the submucosa by a basement membrane. In some embodiments, the bristles which comprise the brush have sufficient stiffness to penetrate said basement membrane and reach said submucosa.

In some embodiments, the apparatus further comprises a handle wherein the handle comprises a distal and a proximal end, wherein the brush is connected to the distal end, and wherein the length of the twisted wire is positioned axially to the brush handle. In some embodiments, the handle comprises a cylinder. In some embodiments, the bristles which comprise the brush are directed outwardly from the twisted wire.

In some embodiments of the apparatus, the bristles which comprise the brush have a tip stiffness, and the tip stiffness of each bristle is between about 0.04 and about 0.2 lbs/inch. In some embodiments, the bristles which comprise the brush have a tip stiffness, and the bristles protrude approximately 0.075 to 0.225 inches from the twisted wire.

Methods to collect cells in epithelial tissue of the body comprise inserting any of the embodiments of the apparatus described above into a body cavity, wherein the body cavity contacts the bristles upon insertion, and collecting cells from at least two layers of said epithelial tissue. In some methods, the bristles collect cells from three layers of said epithelial tissue, said three layers comprising superficial, intermediate and basal layers. In other methods, a basement membrane is located below the basal layer and the bristles penetrate the basement membrane. In some methods, the brush is rotated and collects tissue samples along the length of the brush. Some methods further comprise exerting sufficient pressure on a scrubbing surface in contact with said epithelial tissue to dislodge cells.

In some methods, the body cavity is the endocervical canal and tissue samples are collected from the endocervical canal and the transformation zone. In other methods, the body cavity is the anal canal.

In an embodiment, a disposable kit for diagnostic sampling of the cervix comprises a brush for penetrating an at least two layers of an epithelial tissue of an endocervical canal wherein said brush comprises bristles having sufficient stiffness to penetrate at least said two layers of said endocervical canal wherein said brush is a non-lacerational brush and wherein the bristles collectively substantially form the shape of a cone, and an at least one apparatus for penetrating at least two layers of an epithelial tissue of an ectocervix and collecting a specimen.

The disposable kit may further comprise an at least one sterile container for holding a sample obtained by the brush or apparatus. In some embodiments, a container is provided for each brush and/or apparatus provided. In some embodiments, the container or containers further comprises saline, 10% formalin, or other fixative solution. In some embodiments, the kit comprises an at least one swab suitable for performing a Pap smear. In some embodiments, the kit further comprises an at least one apparatus suitable for taking an endometrial biopsy or sample from a uterus.

For purposes of this summary, certain aspects, advantages, and novel features of the invention are described herein. It is to be understood that not necessarily all such aspects, advantages, and features may be employed and/or achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein. While the description of the embodiments has been primarily directed toward endocervical and or anal canal applications and apparatuses, it is understood by one skilled in the art that the apparatus and method could also be used in other substantially tubular or cylindrically shaped tissues or other shaped tissues or orifices of the body wherein it is desirable to obtain a sample of layers of epithelial tissue in a non-lacerational manner. Furthermore, while description of the embodiments has been in the context of female human use, it is to be understood that the apparatus and method can be used in human males (for non-endocervical canal applications) or mammalian subjects, to include veterinarian contexts.

### BRIEF DESCRIPTION OF THE DRAWINGS

Features and aspects, and advantages of the embodiments of the invention are described in detail below with reference to the drawings of various embodiments, which are intended to illustrate and not to limit the invention. The drawings include the following figures in which:

FIG. 1 is a view of an embodiment of the apparatus with a cone-shaped brush region.

FIG. 2 is a perspective view of the brush region and introducer of an embodiment of the apparatus.

FIG. 3 illustrates a perspective view of the distal handle and brush region of an embodiment of the apparatus.

FIG. 4 illustrates the distal handle and brush region of an embodiment of the apparatus.

FIG. 5 illustrates the brush region of an embodiment of the apparatus.

FIG. 6 illustrates the anatomy of the cervical region of the human female.

FIG. 7 illustrates the distal handle and brush region of another embodiment of the apparatus.

FIG. 8 is a perspective view of the embodiment of the apparatus of FIG. 7.

FIG. 9 is a view of an embodiment of the apparatus with a cylindrical brush region.

FIG. 10 is a perspective view of the brush region and distal handle of an embodiment of the apparatus with a cylindrical brush shape.

FIG. 11 is a view of the distal handle and brush region of an embodiment of the apparatus.

FIG. 12 shows several distal handle configurations of embodiments of the apparatus.

FIG. 13 shows a photographic embodiment of the cylindrically shaped brush head.

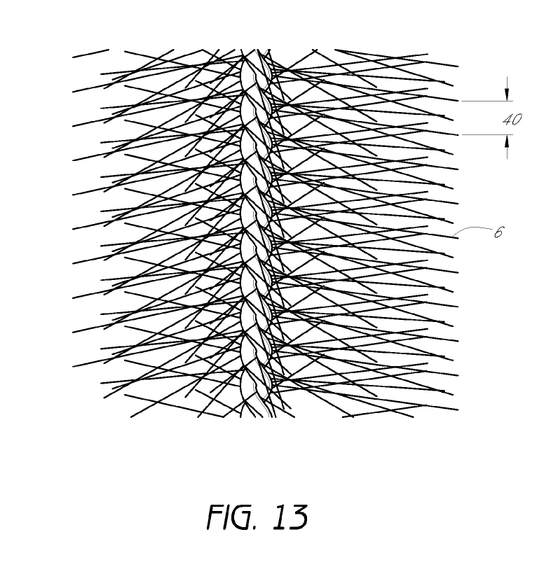

FIG. 14 shows a photographic embodiment of the apparatus with a cylindrically shaped brush head

FIG. 15 shows a photographic embodiment of the apparatus with a conically shaped brush head.

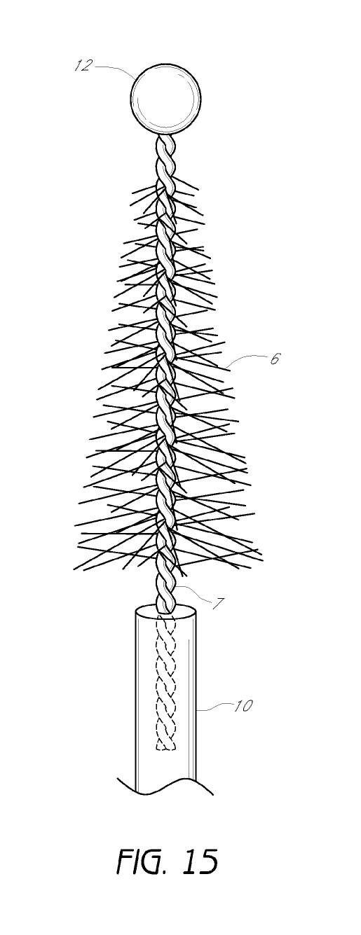

### DETAILED DESCRIPTION OF THE EMBODIMENT

### A. The Anatomy

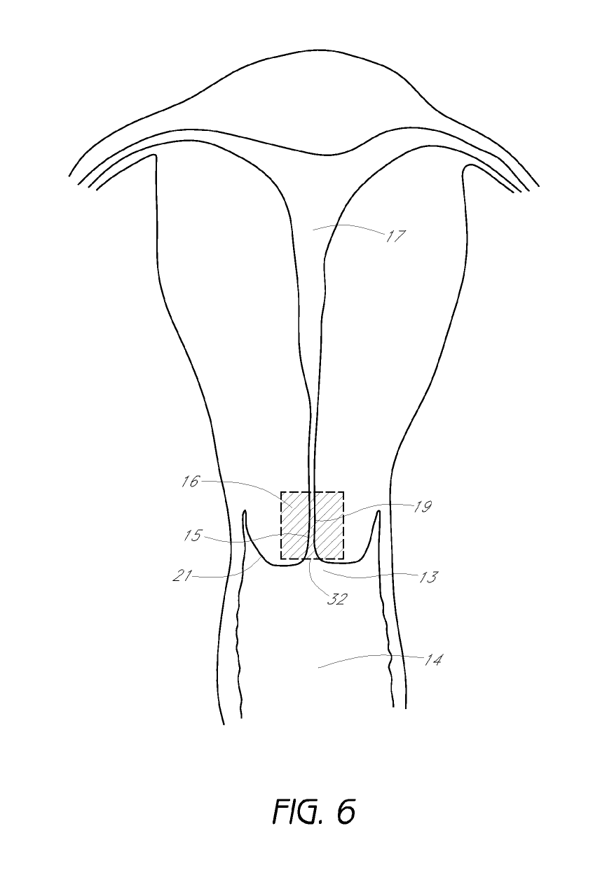

FIG. 6 shows a schematic of the female genital tract, including the vagina 14, ectocervix 13, endocervical canal 16, and uterus 17. The endocervical canal 15 can be seen to be a narrow passageway between the vagina 14 and the uterus 17. The ectocervix 13 is the external portion of the cervix which faces into the vagina and may be directly visualized on gynecologic examination. The opening of the endocervix into the vagina is the external cervical os 32. The transformation zone 19 is the level of the endocervical canal at which the ectocervical tissue changes to the endocervical tissue. The transformation zone is the most common area for cervical cancer to occur. The position of the transformation zone within the cervical canal of an individual patient changes over time, depending on her age, what part of her menstrual cycle she is in, gravidity and other factors. On the proximal side of the transformation zone, the tissue is squamous epithelium and on the distal side, the tissue is columnar epithelium. Because the cells change from columnar to squamous over time (metaplasia), there is an increased incidence of cancer arising in this area. Crosshatching 15 delineates on example of the approximate area to be sampled by the apparatus, and which is inadequately sampled by presently available means. It can be seen that the apparatus may be configured to reach to and/or beyond the transformation zone.

### B. Description of Embodiments

In general, it is recommended that adult women have screening for abnormal changes of the cervix on a yearly basis. This is typically done via a Papanicolaou test (Pap smear), which comprises cytological sampling using a prior art cytological sampling tool such as a swab or soft brush, which collect exfoliated cells from the surface of the ectocervix. This is then analyzed in a laboratory, and is either designated normal, if no abnormal cells are seen in the sample, or abnormal. If it is abnormal, the patient typically undergoes a procedure called colposcopy, in which the provider uses magnification to look directly at the ectocervical region, notes the presence of grossly visible lesions, often aided by the application of acetic acid and takes samples of the lesions, either with curette, punch, or other traditional means, or with a SpiraBrush®. At the same time, the provider typically takes a specimen from the endocervical region to screen for presence of abnormality there. At the present time, means for sampling the endocervical region are limited to cytological sampling with soft, non-abrasive brushes or swabs or histological sampling using curettes. Cytological sampling of the endocervical region has the same limitations as cytological sampling of the ectocervical region.

With respect to histological endocervical tissue sampling, the primary devices in use today are endocervical curettes, such as the Kevorkian-Younge endocervical curette. These are typically stainless steel implements with a narrow end suitable for fitting into the endocervical canal and obtain a sample by scraping from distal to proximal along the endocervix, such that a strip of tissue is obtained. This is often very painful for the patient. Furthermore, it is difficult to control the depth to which tissue is removed, so that either inadequate or excessively thick samples may be taken, and laceration and/or bleeding are common. Also, the sample obtained only comes from a small arc of the total endocervical surface, so even a large area of abnormality can be missed. Another difficulty experienced with the endocervical curettes of the prior art is in retrieval of the tissue sample once obtained. Frequently, some or the entire sample is damaged or not readily retrievable without a separate step and/or implement.

Therefore, it is clearly advantageous to provide an apparatus for obtaining a histological sample from an endocervical canal in which multiple layers may be non-lacerationally obtained without requiring direct visualization and in which the surface area of the endocervical canal is representatively sampled over a large percentage of its area. Since many lesions, in their early stages, may not be detectable without obtaining multiple layers of epithelium, at times down to the basement membrane, it is clearly advantageous to provide an apparatus which can obtain multiple layers for examination. Furthermore, it is clearly advantageous to provide a method for using the apparatus to obtain a specimen.

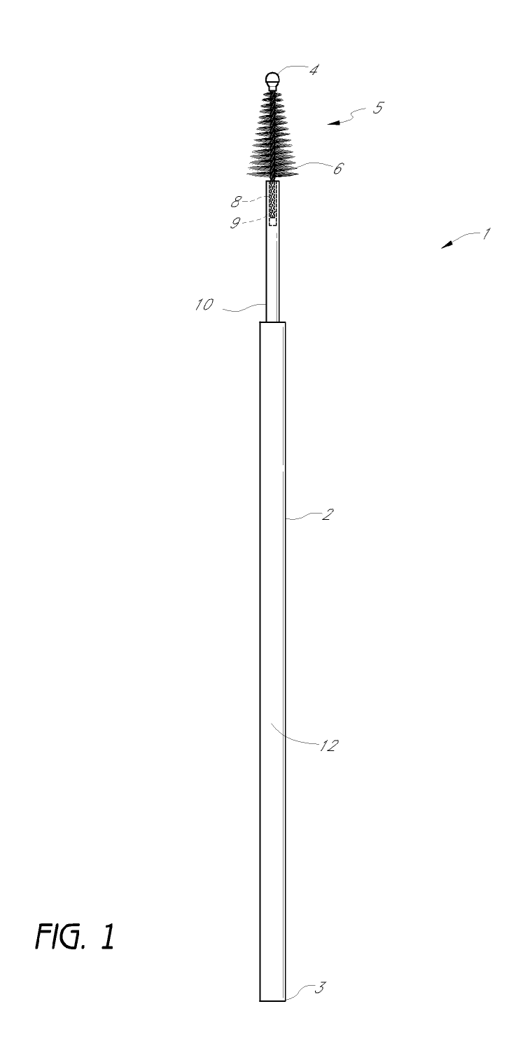

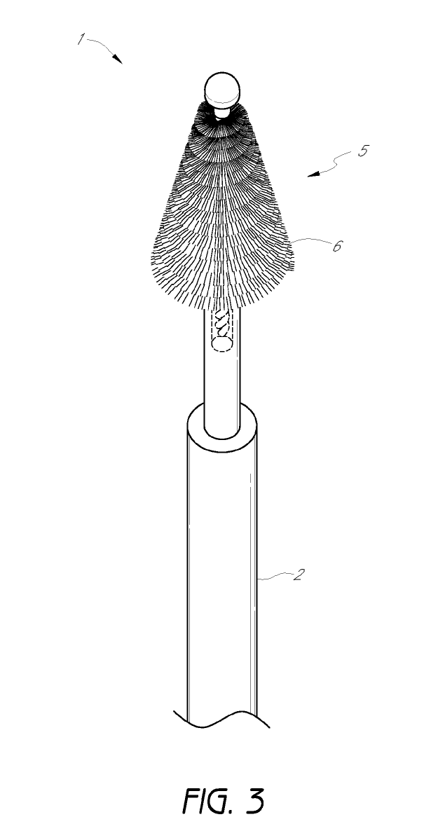

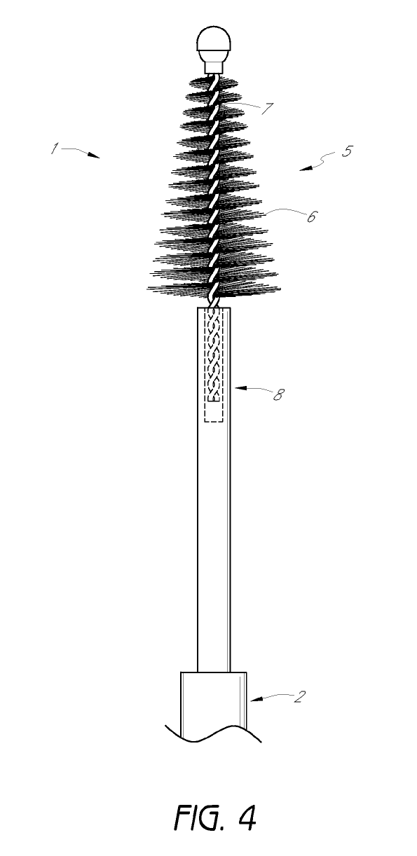

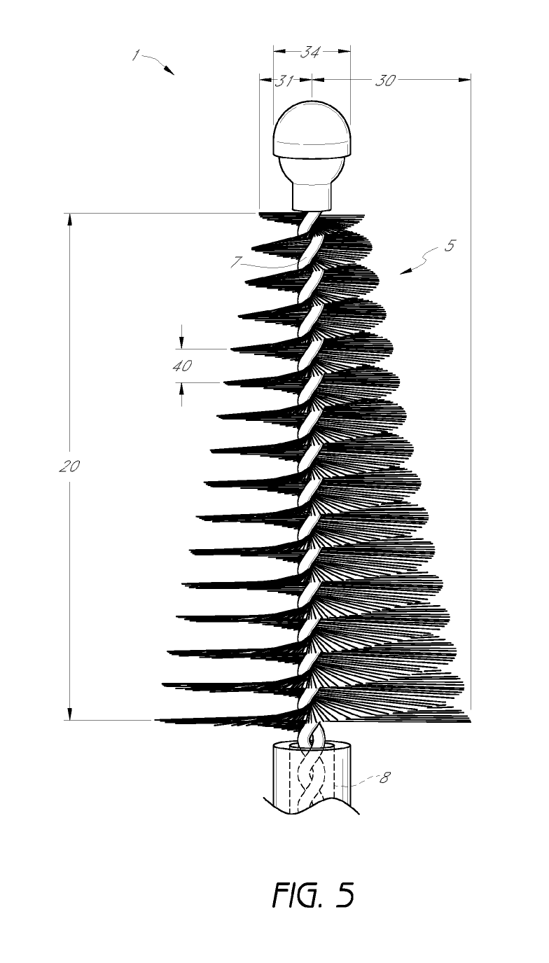

As seen in FIGS. 1-5, the apparatus 1 may have a distal end 4 and a proximal end 3. The apparatus 1 may include a handle or elongate member 2 comprised of a handle body 11 and a neck 10, as well as a brush head 5, comprised of twisted wire 7 holding the bristles 6, which may be shaped, for example, to conform to the shape of endocervical canal, and a rounded or cone-shaped tip 4 on the distal end. The total length of the device may be approximately 10 inches in some embodiments. An advantage of the length may be that it is of a length to allow the user to comfortably manipulate the proximal handle from external to the vagina during sampling, while it limits the distal extent to which the user may insert the apparatus due to contact with the opening of the vagina. In other embodiments, the length of the device may be 7, 9, 11 or 13 inches.

### C. The Handle

The handle 2 is designed for gripping by a user, and is of a sufficient length to allow the user to manipulate the device within a body cavity from a location just outside the body. The handle is semi-rigid so as to assist in reaching the target tissue notwithstanding difficult angles or narrow passages.

The handle 2 can be constructed of a plastic, such as polypropylene, or any other suitable semi-rigid material. The handle may further comprise a body portion and a neck portion. In some embodiments, the body portion and the neck portion may have differing diameters. The neck portion diameter may be smaller than the body portion diameter. The neck diameter may range from 0.05 to 0.3 inches, and in some embodiments between 0.1 and 0.2 inches, with a diameter in one embodiment of 0.13 inch. The diameter of the handle body may range from 0.7 to 0.5 inches, and in some embodiments between 0.15 to 0.3 inches, with a diameter in one embodiment of 0.22 inch. The narrower neck may increase the distal flexibility of the handle and to better accommodate the female anatomy and allow entrance into and passage along the endocervical canal 15 in line with its axis, which has an anterior and proximal orientation with respect to the vaginal opening and cavity. The neck section may range in length in some embodiments from approximately 0.5-5 inches. This may allow the narrower neck section to traverse the vaginal area from the vaginal opening to ectocervical area. Alternatively, a shorter neck area may be preferable to increase stiffness of the handle for manipulation. The increased diameter of the body may assist in maintaining sufficient stiffness proximally to allow the user to exert control over manipulation of the handle. As discussed below, the increased diameter of the handle body may also allow for improved grip and rotational control of the apparatus during sampling. In other embodiments, the handle has a constant diameter along its length.

The handle body may be solid throughout, or may contain a hollow section 8 approximately 0.5 inches long in the distal end of the handle neck 10 to accommodate the insertion of a portion of the apparatus such as the base of the twisted wire brush head 9. In some embodiments, the hollow section 8 can be from 0.2-1.5 inches long to accommodate the base of the brush head. In other embodiments, the brush region is connected to the handle by other means. In some embodiments, the base of the brush extends through a hollow section in the entire handle. In other embodiments, the support for the brush head itself comprises the handle neck and/or the handle body. In still other embodiments, the wires are are secured to the handle via a coupling feature.

In some embodiments, the handle body 11 has at least one area with cross-section that is substantially circular such that the elongate member 2 may be readily twirled between the thumb and forefinger while inserted within the endocervical canal 15, or other body orifice. This rotational ability will also be effective in the brush bristles 6 passing through the transepithelial layers to retrieve cells. In other embodiments, the proximal end of the handle may comprise a loop to be held between, for example, the finger and thumb of the operator during insertion and rotation. In still other embodiments, the proximal end may comprise a flattened section either along the shaft or at a proximal end, such that the handle may easily be rotated in 180 degree increments using a finger and thumb, and the number of rotations tracked. Other configurations of the handle for control and manipulation of the apparatus are also contemplated and would be obvious to one skilled in the art.

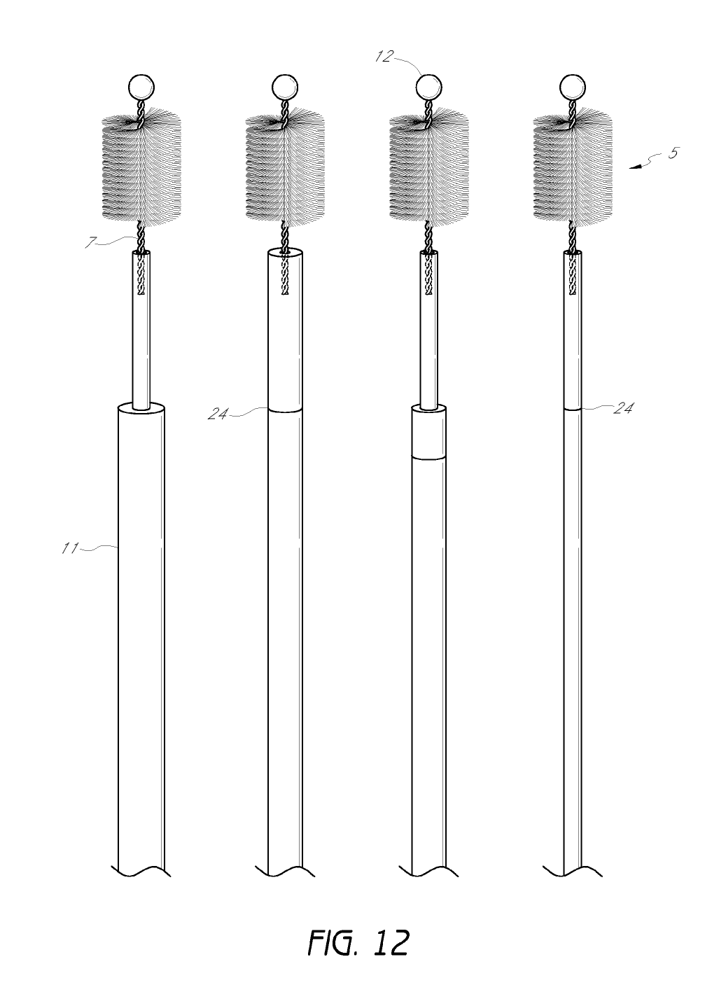

The region between the handle body 11 and neck 10 may be scored 24 (FIG. 12) to allow the handle neck 10 and brush head 5 to be removed with sufficient lateral pressure to snap the scored neck from the handle. In other embodiments, the scoring is on the middle portion of the handle neck. In still other embodiments, the neck portion may be detachable from the handle body by screwing off. This allows the brush head and accompanying sample tissue to, be non-disruptively and without contamination, placed into a container to be sent to a laboratory, where removal, fixation of the tissue, and examination can be accomplished. The lateral force required to nap the scored neck from the handle may be in the range of 20-60 pounds, sometimes in the range of 30-40 pounds, or in the range of 35 pounds.

In some embodiments, the handle may be reusable and designed to accept a distal brush portion which is disposable. The connection may involve snap on, frictional fit, mechanical interfit, or screw on configurations, among others. In some configurations, fixation is accomplished through the distal portion fitting over the handle segment, while in others, the distal portion enters a hollowed out area of the handle.

### D. The Brush Head

At the distal end of the handle neck 10, the device may comprise a brush head 5. The brush head's basic form can comprise, in some embodiments, three parts: twisted wire 7, bristle section 6 and an inserter tip 12. In other embodiments, it may comprise more or fewer parts. For example, in some embodiments, the twisted wire may be replaced by a different central longitudinal member which supports the bristle section, and the distal end may be adapted for atraumatic insertion of the apparatus. In still other embodiments, for example, there may be additional parts such as a coupling piece for attaching the twisted wire to the handle, either releasably or non-detachably.

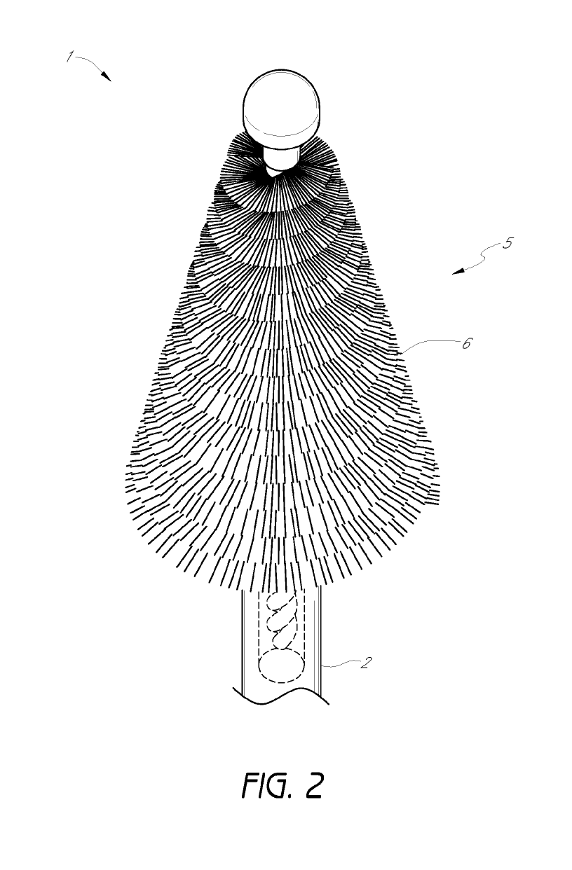

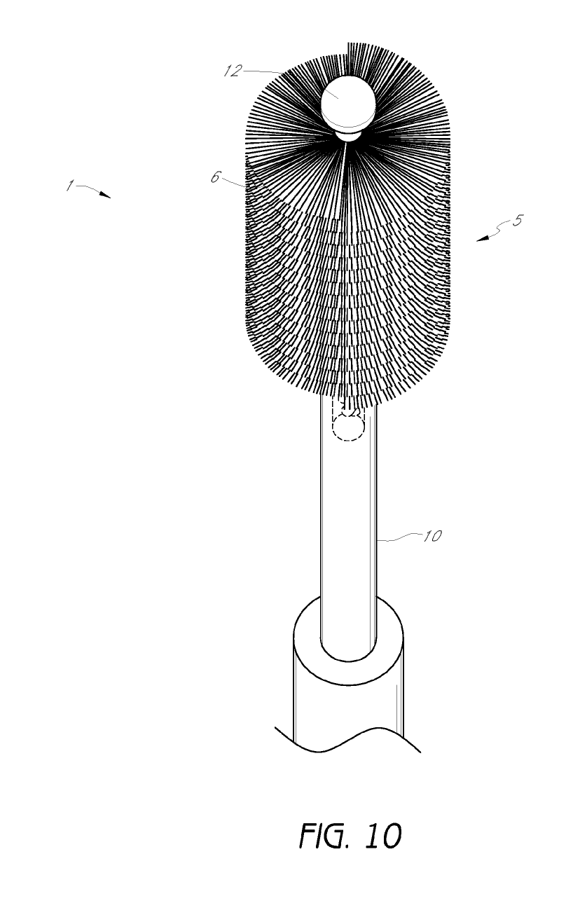

The brush head 5 may be formed from one or more twisted or braided wires, cables, or backbone 7, and may be in various shapes to accommodate the particular orifice in which it is used such as in the shape of a cone (FIG. 2, for example), frustoconical, or cylinder (FIG. 10, for example). The twisted wires 7 may be secured to handle neck 10 in a recess in the distal end of the handle neck 10. The brush 1 can be formed from conventional twisted wire brush construction. In some embodiments, the total length of the twisted wire 7 is approximately 1.5 inches, with approximately 0.5 inches inserted in the hollow section of handle 8, and approximately 1 inch exposed as part of the cone or cylinder, such as about 0.8-1.2 inches in length. In other embodiments, the twisted wire may range from 0.5 inches to 3 inches in length in the portion extending from the distal end of the handle. In still other embodiments, the wire may extend proximally to comprise the handle, and may be 5-15 inches in length.

In some embodiments, the twisted wires are made of surgical steel, while in other embodiments, the wires can comprise a softer metal suitable for sterilization and use within the body, polystyrene, polyethylene or other suitable plastics. The diameter of the wires used can vary based on the stiffness and spacing of bristles desired and may range from 0.02 inch to 0.1 inch. A single wire may be twisted upon itself or multiple wires may be twisted together to secure the bristles in position.

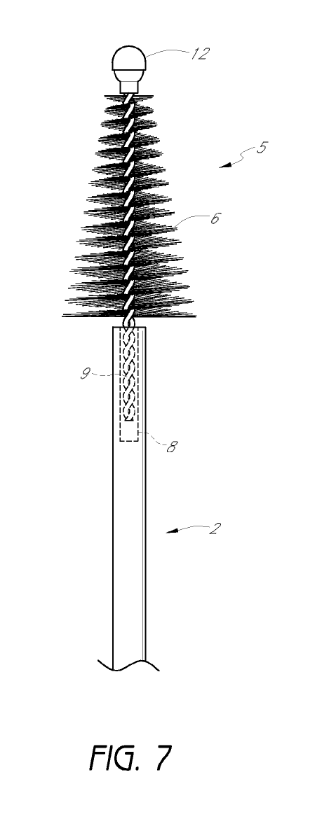

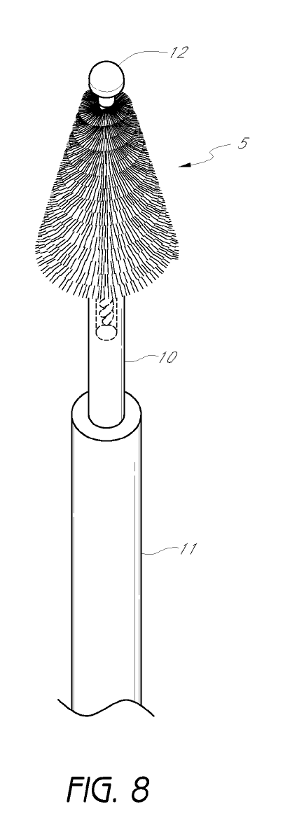

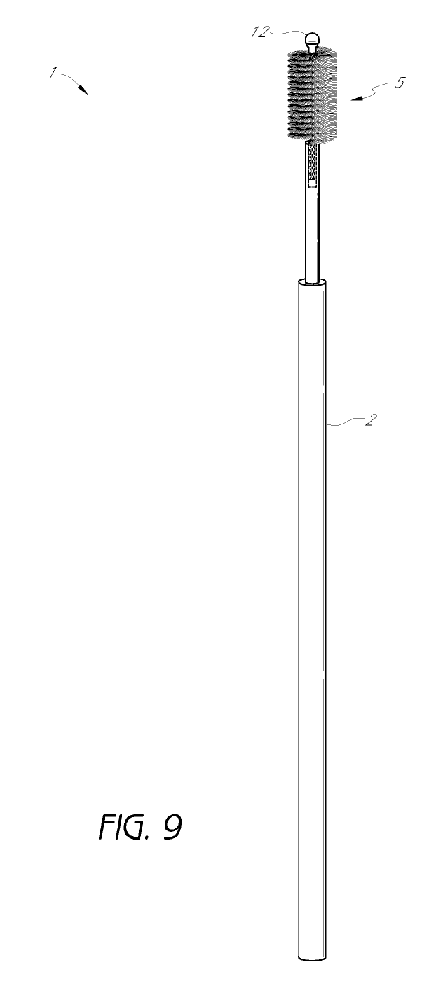

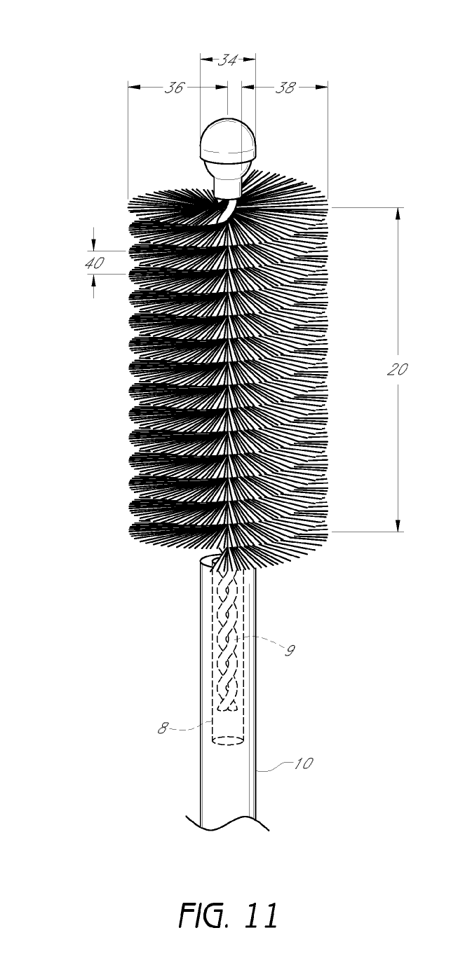

The twisted wire portion 7 may stand straight out from the handle neck 10 with the rounded or cone-shaped tip 12 on the end, that will facilitate entry of the brush into the endocervical canal. The bristles 6 are inserted, being held in place by the tension of the twisted wire 7. From the rounded or cone-shaped tip to the base of the twisted wire, the bristles may be arranged from smallest to longest, thus forming a cone shape (see FIGS. 1-5, 7, 8), or may be of uniform length, thus forming a cylindrical shape (see FIGS. 9-11). In other embodiments, the bristles may be fixed into a central support structure without being twisted.

The length of the brush head 20 is designed in some embodiments to traverse the endocervical canal from at least the cervical os 22 to the transformation zone 19 to permit sampling of the entire region. The length in some embodiments may be from 0.5 inch to 3 inches. In other embodiments, the length may range from 0.8 to 1.2 inches. Multiple brushes may be provided to accommodate differing anatomies and ensure adequate sampling. This provides significant advantages, particularly since the sampling is done blind. Insertion of the brush until the proximal bristles have just entered the os provides that the distal bristles extend to or just beyond the transformational zone. As the transformational zone is the most frequent origin of cancer within the endocervical canal, it is particularly useful to ensure sampling to at least this depth. Some methods of sampling the endocervical canal with the apparatus comprise inserting the brush until the proximal bristles have entered the cervical os, then rotating the brush in position before withdrawing the brush. The specimen is collected by rotating the brush rather than utilizing longitudinal movement as may be necessary if the brush head portion were not sufficiently long. Once the proximal bristles have entered the os, it is more difficult to gauge further depth of insertion, and there would be a risk of over-insertion distally, resulting in potential unwanted tissue damage or specimen contamination with, for example, endometrial tissue. Similarly, if the brush head were too long, the provider would have to guess where to limit insertion or potentially insert to the full extent of the brush. In this case, damage could result to tissues distal to the preferred sampling zone and/or contamination could occur with endometrial or ectocervical tissue. In some embodiments, the brush head may be provided in suitable length for obtaining specimen from the anal canal.

The brush head 5 may be provided with a plurality of bristles 6. The bristles 6 may be approximately 0.25 to 0.55 inches from tip to tip, and may have a protrusional length of 0.075 to 0.225 inches from the lateral aspect of the twisted wire to a tip of a bristle. In other embodiments, the tip to tip length may range from 0.15 to 1.8 inches and the protrusional length may range from 0.05 inch to 0.75 inch. The tip to tip length of the bristles may determine in part the initial pressure of the brush against the canal walls and may have an influence on the bristle's resistance to being dislodged from the tissue surface. The protrusion length may be important in determining stiffness, for example, lateral tip protrusion stiffness, as the ratio between the bristle diameter and the protrusion length may contribute to the lateral tip deflection force of the bristle. The bristles 6 are approximately 0.006 inches (0.16 mm) thick in one embodiment, based on the diameter of a bristle's cross-section. In other embodiments, the bristle thickness may range from 0.001 to 0.1 inch, while in others the range is from 0.003-0.001 inch. Brushes may be made in various sizes to accommodate the size of the endocervical canal of an individual, as the endocervical canal ranges in size depending on the patient. Specifically, there may be at least two diameters of the brush for use in endocervical sampling, one for young adults or those whose endocervical canals are generally narrower in diameter, and one for mature women. Further embodiments of the brush include longer bristles suitable for use in anal sampling applications. The diameter of a brush in such embodiments may range from 0.25 inch to 1.5 inches from tip to tip. In other embodiments, the diameter may range from 0.4 to 0.8 inches from tip to tip. These diameters accommodate the dimensions of the anal canal more accurately.

The length of the stiff bristles of the brush may tapered from tip to base, such that the brush is cone-shaped, while still maintaining a balance between: 1) the requirement to keep the bristles stiff enough to grind the tissue into its cellular components during rotation; 2) the requirement that the bristles be long enough to trap the removed cells and 3) the requirement that the bristles be soft enough to bend. In embodiments of the apparatus in which the brush is substantially cone shaped or partially cone-shaped (frustoconical), the length of the bristles from center of the wire to tip may for example range from a length 31 at the distal end of the brush (L1) of 0.075 inch to a length 32 at the proximal end of the brush (L2) of 0.225 inch. In other embodiments, L1 may range from 0.005 inch to 0.225 inch and L2 may range from 0.075 inch to 0.4 inch. The increase in diameter from distal to proximal may be a relatively smooth one, or accomplished in a series of steps. The bristles at the proximal end may have a different cross-sectional diameter than the bristles at the distal end to maintain a relatively constant lateral tip deflection stiffness along the length of the brush. The ratio of L1/L2 may in some embodiments be approximately 0.333. In other embodiments, the ratio may range from 0.25 to 0.45, while in still other embodiments, the ratio may range from 0.1 to 0.9. An advantage of these ratios can be to fit the anatomy of the endocervical canal while still maintaining desirable stiffness and lateral tip deflection characteristics of the bristles. This may assist in detaching tissue to a substantially similar depth both proximally and distally in the canal.

In embodiments of the apparatus in which the brush is cylindrically shaped, the center to tip length of the bristles 36 may for example range from 0.080 inch to 0.45 inch, in other cases from 0.175 to 0.35 inches, and in some 0.23 inch. Multiple brushes may be provided with different tip to tip diameters to accommodate varying diameters of the cervix. It is advantageous to provide a brush with bristle length which is optimal for the diameter of the endocervical canal. When a sample is taken from the ectocervical region, pressure can be exerted distally along the longitudinal axis of the handle to modulate the initial force exerted by the bristle tips on the tissue as they contact the tissue generally perpendicular to the surface plane. In contrast, in a substantially cylindrical space such as the endocervical canal or anal canal, longitudinal pressure along the handle controls the depth of insertion, but is not useful for modulating the initial pressure on the tissue by the bristle tips. However, the length of the bristles in relation to the diameter of the canal, in conjunction with bristle stiffness, will help determine the depth of initial penetration and/or pressure applied to the surface. If the tip to tip diameter of the bristles is too short or the bristles insufficiently stiff, the bristles may slide over the surface of the tissue on brush rotation without obtaining a specimen other than exfoliated cells. If the tip to tip diameter of the bristles is long or the bristles excessively stiff, the bristles may, for example, lacerate the tissue as the apparatus is rotated.

Another measurement which may be important to the function of the brush is the protrusional length 38 of the bristles, which is the length of bristle measured from the point at which the bristle emerges from the central twisted wire or other central support to the tip of the bristle. This defines the length of bristle upon which bending forces are exerted. The ratio of protrusional length to bristle cross-sectional diameter may determine the lateral tip deflection stiffness. In embodiments, the protrusional length of the bristles may for example range from 0.075 inch to 0.4 inch, in other cases from 0.15 to 0.3 inches, and in some 0.225 inch.

In some cases, the stiffness of the bristles determines the ability of the bristles to penetrate the tissue to a sufficient degree to obtain a histological tissue sample in contrast to a sample merely containing exfoliated cells. Several factors affect the stiffness of the brush: a) the extent to which the wire is twisted to keep the bristles in place; b) the thickness of the bristles, and c) the length of the bristles. In some aspects, the length of each bristle or the extent that they protrude from the wire, contributes to the stiffness to a greater degree than how tightly the wires are twisted or the thickness of the bristles. The stiffness may be better identified as cantilever or lateral tip deflection stiffness. The lateral tip deflection stiffness may determine the number and depth of cells detached on each pass of the brush. An increase in lateral deflection stiffness will increase the lateral shear force exerted on the contacted tissue, and perhaps result in a lacerative effect rather than an abrasive one. Conversely, a decrease in lateral deflection stiffness will decrease the shear force, perhaps resulting in loss of contact between the tissue and the bristle rather than detachment of a layer of tissue. Therefore, it can be advantageous to achieve a lateral tip deflection stiffness of the bristles which is optimal for use in the endocervical region, whereby the cells are removed with passage of the brush, but laceration is avoided. Also, it may be advantageous to provide a brush in which the lateral tip deflection stiffness is such a passage of the tips against the squamous epithelium proximal to the transition zone results in detachment of a layer or layers of cells, while the lateral tip deflection stiffness is less than that required to detach the columnar cells distal to the transition zone.

Thus, the ratio of bristle thickness (cross-sectional diameter) to bristle protrusion length (the TPL ratio) can be important for achieving a lateral tip deflection stiffness which permits sampling through at least two layers of endocervical epithelial tissue without lacerating the tissue. If the TPL ratio is too low, then the bristles can be too flexible, and, instead of achieving a histological sample of at least two layers of epithelial tissue, only exfoliated and surface cells/tissue will be obtained. If the ratio is too high, then the bristles can be likely to lacerate the tissue rather than detaching layers of cells. In some embodiments, the ratio is between 0.01 and 1, in others between 0.022 and 0.2, and in still others between 0.04 and 0.1. In some embodiments, the TPL ratio is no less than 0.02, in some embodiments, the TPL ratio is no less than 0.025, in some embodiments the TPL ratio is no less than 0.03. In some embodiments, the TPL ratio is no greater than 0.035, in some embodiments the TPL ratio is no greater than 0.04, in some embodiments the TPL ratio is no greater than 0.06, in some embodiments the TPL ratio is no greater than 0.08.

Although in some applications it may be desirable for the brush to penetrate a keratinized layer to obtain a tissue sample from below a layer of keratinized tissue, with respect to endocervical sampling, the bristles may have an intermediate level of stiffness that is below that required to penetrate keratinized tissue but above the stiffness for merely obtaining a non-histological sample of exfoliated cells. Such stiffness may allow collection of samples in the more sensitive endocervical region without being overly invasive and damaging to the sensitive endocervical tissue, but sufficient to penetrate the ectocervical tissue so that a histological sample may be obtained from the ectocervical region and the transformation zone between the endocervical and ectocervical regions. A non-invasive brush may cause some bleeding when used, but does not cause excessive bleeding such that it compromises the quality of the histological sample, and does not cause permanent damage such as scarring, stenosis, or adhesions of the endocervical canal or contribute to an incompetent cervix.

Thus, the bristles may have a lateral deflection force of between 0.04-0.2 lbs/inch. Brushes having an intermediate level of stiffness, greater than a conventional Pap smear brush but less than one that penetrates keratinized tissue, may have a stiffness of 0.06-0.1 lbs/inch. This allows for sampling of multiple layers of epithelium without excessive damage to the sensitive underlying tissue. In some embodiments, an apparatus may be provided with greater lateral deflection stiffness to be used in the setting of a known keratinized lesion or when deeper penetration is desired.

In some aspects, the bristles may be covered by a sheath, such as one described in U.S. Pat. No. 5,713,369, to avoid insult to the tissue upon entering the endocervical canal. For instance, in some uses, once the sheath-covered bristles are placed to the appropriate depth into the endocervical canal, the sheath may be retracted and the brush may be rotated to obtain a sample. The use of the sheath during insertion may additionally prevent contamination of the specimen with cells or tissue from the cervical os or ectocervical region. After sampling, the sheath may be employed to cover the brush containing the sample, so that the brush can be removed without further insult to the endocervical canal and without contamination of the sample from contact with the ectocervix or vagina.

In some aspects, the brush head 5 is designed to disrupt the tissue of a lesion and penetrate beneath the superficial layer of the epithelium to sample multiple layers such as at least three epithelial layers, which is especially useful for obtaining histological samples from the endocervical region at the transformation zone. The brush can penetrate through transepithelial tissue layers to retrieve cells. It may be preferable to have a plurality of scratches or furrows in the tissue from the brush, one of which will penetrate the basement membrane over a substantial area of the lesion. In additional aspects, each stroke penetrates a little so that the depth of penetration can be controlled by the appearance of light bleeding at the os. Since, in the endocervical region, it is not possible to obtain the sample under direct visualization, it is especially advantageous that the brush be able to penetrate a layer at a time into the epithelium. This permits the operator to control the depth of the specimen taken by the number of passes made by the bristles.

Further, an advantage and feature achieved with the brush is that a rather large area within the endocervical canal is subject to the action of the brush, which increases the tissue collected, in contrast to conventional endocervical curettage. In addition, unlike situations in which the lesion is visible such as a lesion in the ectocervical region, an endocervical lesion may not be visible to the person obtaining the sample; therefore obtaining a histological sample over a large surface area will advantageously increase the ability to collect a sample that is sufficient to detect an abnormal or cancerous lesion. In contrast, endocervical curettage yields a much smaller sample from a smaller portion of the total surface area. Thus, a lesion on an unsampled region may easily escape detection. Also, as discussed above, particularly in a situation where the operator has no visual feedback, it can be difficult to control the depth of the sample taken, and there is a risk that the sample is excessively superficial and not adequate for examination. There is also a risk that the sample taken is deeper than intended, and can cause excessive bleeding, pain, and/or scarring.

The configuration of the brush allows for limited space between the points of abrasive contact upon brush rotation, while maintaining enough separation between the bristles to trap a clinically effective amount of cells. The limited space between the points of abrasive contact upon brush rotation ensures that a large portion of the surface area of the endocervix is sampled during use, and even relatively focal lesions are likely to be sampled. The ability to trap the effective amount of cells is particularly advantageous because the confined space of the endocervical canal can make it difficult to effectively withdraw a specimen, as, for example, when a curette is used.

The types of bristles contemplated include a variety of potential tip types. In embodiments, the bristles are each stiff or semi-rigid. In some embodiments, the bristles are preferably made of Tynex® brand nylon laid in a double or single layer. The Tynex® brand bristles have their own cantilever stiffness which may be at a modulus of 500,000 psi. Alternatively, triple row densities may be used. Other types of nylon and other appropriate materials may be used, such as soft metals, plastics, polystyrenes, and any material which can form a bristle of the appropriate size and stiffness for employment in an apparatus. The front edge or tips of the bristles may provide good scraping or cutting surfaces. The front edges or tips of the bristles may describe tiny “hooks.” These scraping or cutting surfaces may help to dislodge the cells from the surrounding tissue to be collected on the bristles. The sharp edges may be designed to not dig too deeply into the epithelial tissue and thus avoid severe injury. In cross section, alternate brush structures may include ones in which the front edges of the bristles are squared or “file card wire brush type edges” which are aligned. In embodiments with squared bristles, the tips may form non-penetrating scraping edges and even generate negative rake angles when the bristles bend over. It is preferable to have a large number of such bristles to spread the pressure as the brush structure is being used.

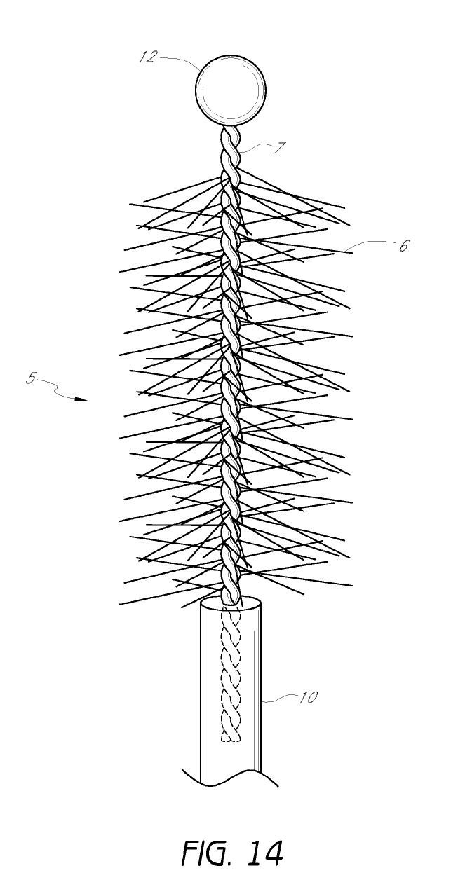

In some embodiments, the number of bristles may be in the range of 400/1600/inch of brush length. In some embodiments, where the brush length is 0.5 inches, the total number of bristles may be 200-800, in some 200-450, in others 650-800. In other embodiments, where the brush length is 1.0 inches, the number of bristles may range between 400 and 1600. The distance between turns of bristles 40 may range between 0.035 and 0.2 inch, or may range between 0.05 and 0.12 inch. This density not only ensures that the pressure is distributed so that pressure is advantageous spread along the area to be sampled, but also ensures that, as the apparatus is rotated, bristle tips contact the area so that little area is left unsampled and yet there is adequate spacing for the cells thus sampled to move toward the wire core and become trapped between the bristles. The bristles 6 may be arranged substantially perpendicular to the longitudinal axis of the twisted wire 7. Alternatively, they may extend either distally or proximally at an acute angle to wire 7. In yet another alternative, the bristles 6 may be arranged such that some extend distally and others proximally at angles from the wire as seen in FIG. 14, for example. The angles may additionally vary amongst the bristles. This may be advantageous for collection and retrieval of the specimen, as cells may become more securely trapped between the bristles.

Another component of the brush head is an insertional tip 12 on the distal end of the apparatus. The endocervix describes a basically cylindrical shape with relatively soft walls. The twisted wire or other longitudinal support structure from which the bristles radiate thus has a distal tip which is stiff and narrow, and likely to cause laceration or puncture of sensitive endocervical structures during insertion and/or withdrawal of the apparatus. Therefore, it is advantageous to have an insertional tip 12 to protect the tissues and side walls of the cervical canal by distributing a force across a greater surface area thereby preventing puncturing of the side wall tissue and/or the creation of false passages and/or bleeding and/or infection. This tip 12 also can create a temporary dilation in the cervical walls in advance of the bristles, thus less bending or deformational force can be exerted on the bristles during insertion. This dilation effect may be particularly useful in a situation in which the cervical os is stenotic, such as in very young and post-menopausal women, or those with scarring from previous insult. The insertional tip 12 may be spherical, rounded, or substantially conical in shape. The tip may be made of plastic or silicone or other flexible, bendable, deformable, or suitable material. Alternatively, the tip may be made of polystyrene, rubber, polylactic acid (PLA), or other suitable materials. Alternatively, the insertional tip 12 can be substituted with only dipping the distal end of the wire of the brush head in a material suitable for blunting the wire tip. Materials can include but are not limited to polystyrene, rubber, polylactic acid (PLA), or other suitably bendable, flexible, or deformable materials. In some embodiments, the tip has a cross sectional diameter 34 from 0.05 inch to 0.25 inch, and in some embodiments between 0.08 and 0.2 inches, and in some embodiments, the cross-sectional diameter is 0.11 inch. The diameter of the insertional tip should be sufficiently wide to distribute the force from longitudinal pressure during insertion such that the endocervical wall is protected from puncture, and sufficiently narrow in comparison to the tip to tip length of the bristles that it does not separate the tissue such as to interfere with the ability of the first row of bristles to penetrate layers of epithelial tissue and obtain a sample. For example, in some embodiments, a ratio between the diameter of the insertional tip and the diameter of the bristles from tip to tip may range between 0.2 and 0.8, often between 0.4 and 0.6.

### E. Method

Rotation of the bristles against the endocervical canal walls results in the scraping detachment of cells from multiple layers of the epithelium. Each pass of the bristles over an area results in the detachment of a further layer of cells. The detached cells become collected between the stiff bristles and are trapped there. These cells can then be inspected by a suitable laboratory. This is advantageous over traditional curettage, which, after detaching a strip of tissue, often has difficulty retrieving the specimen.

The method is particularly advantageous due to the fact that, as no laceration of the epithelium is required, the discomfort experienced by the patient is considerably less than that of an endocervical curettage, and is generally minimal. Similarly, because each rotation of the brush penetrates slightly deeper into the epithelium, the operator is able to control the depth of penetration better than with curettage, while ensuring that an adequate sample is obtained.

Further, this method effects sampling over a large area, in contrast to the method of the endocervical curettage which is confined to a narrow strip of tissue. It is particularly important to obtain cells from as large of an area as possible, to minimize the possibility of missing a lesion. Since there is no opportunity to view the endocervical canal directly, as is possible to do with the ectocervix, it is not possible to visually confirm that representative sampling has occurred, nor is it possible to ensure that all suspicious areas are sampled. Therefore, a method which predictably results in a large area being sampled to a consistent depth, is particularly useful in the evaluation of the endocervical canal.

A method of use of the apparatus can comprise obtaining visualization of the cervix and cervical os and passing the tip of the apparatus into the os with longitudinal pressure until it reaches a depth in which the proximal row of bristles has passed into the os. The handle of the brush is then rotated in only one direction (clockwise or counterclockwise) through one to five turns, in some instances two to four turns, and in instances, three turns, while maintaining the insertional depth of the apparatus. Alternatively, rotation may occur in alternately clockwise and counterclockwise directions. A small amount of blood may be observed at the entrance to the os and signal that an appropriate depth has been reached. The apparatus is then withdrawn from the cervix and removed from the patient. Rotation of the bristles permits scraping detachment of cells from multiple layers of the epithelium. The cells, as they are detached, become collected between the bristles and are trapped there, permitting retrieval upon withdrawal of the apparatus.

In some applications of the method, prior to endocervical sampling with the apparatus, colposcopic examination is performed, and samples are taken of any visible lesions or lesions revealed by the application of acetic acid on the ectocervix. In some applications of the method, Pap smear is performed during examination and prior to endocervical sampling. In other versions of the method, endometrial sampling is also performed in the same session.

After endocervical sampling and removal of the apparatus, the distal portion of the brush may be removed, either at an location of scoring, by lateral pressure on the neck/body junction, or by unscrewing a distal portion, and placed into a container for transport to a laboratory. The container may contain formalin, saline, or other fixative. The proximal handle section may be discarded.

In some methods, a brush for sampling the endocervical canal comprises a sheath or similarly functioning structure, which in an extended state overlies the bristles of the brush and in a retracted state remains proximal to the brush. During insertion and removal of the brush, the sheath may be extended to prevent the brush from contacting vaginal, ectocervical, cervical os, or endometrial tissue. This may be advantageous in preventing abnormal cells from these tissues from entering the sample and providing assurance that the cells in the specimen come only from the targeted area, for example, the endocervical canal. In other methods, the user may remove exfoliated or loose surface cells from the cervical os and surrounding ectocervical tissue using a swab, brush, or other similar device before insertion of the endocervical brush as disclosed herein to prevent the ectocervical or cervical os cells from being sampled by the brush.

A method of obtaining a transepithelial sample in which a brush as described herein is inserted into—a generally cylindrically shaped area of tissue, sometimes without direct visualization, then rotated while maintaining the longitudinal insertional placement, provides a drilling action on the surrounding surface, with slightly deeper penetration on each successive rotation. This drilling presents the ability to thoroughly sample all layers of the epithelium without the necessity of performing a surgical laceration. Specifically, the methods of obtaining a transepithelial sample from a tissue canal provide a unique combination of: 1) sufficient manual pressure on the handle of the device to maintain the depth of insertion of the brush within the canal; 2) keeping the sharp bristle edge in contact with the epithelium circumferentially and, 3) rotation of the device, which results in a superior “drilling” action in the epithelium which has previously been unknown in the art of endocervical curettage. Furthermore, the method effects sampling over a much greater area of the endocervical canal than was previously possible with traditional methods, thereby significantly reducing the chance of missing a lesion, which will likely reduce morbidity and mortality associated with cancer or other pathology arising in the endocervical canal. While the methods of obtaining a transepithelial sample are disclosed primarily in relation to an endocervical canal, they apply as well to other areas of tissue in the body including, among others, the anal canal, the throat, the esophagus, the trachea, the bronchi, and the nasal passages. The methods may be particularly suitable when the tissue to be sampled is an elongated structure with a surface area describeing an interior surface of a lumen.

### F. Kits

It is contemplated that kits be provided to medical providers to ease the evaluation of cervical pathology. For example, colposcopic examination is generally performed after obtaining an abnormal Pap smear. On colposcopy, most frequently 2-4 areas of abnormality are identified either on direct vision or on application of acetic acid. At that point, sampling is performed of each focus of abnormality and a separate sampling is taken from the endocervix to determine whether there are further abnormal areas in the endocervical canal. It would be advantageous to supply kits with an endocervical sampling brush as described herein and at least one, perhaps two to three brushes for sampling ectocervical lesions, and sometimes four to five brushes for sampling ectocervical lesions. Some kits may have one or more disposable or reusable punches for sampling ectocervical lesions in addition to or instead of some or all of the ectocervical brushes. Some kits may also have an at least one container sized to accept the distal end of the sampling apparatus, some kits with one container for each sample. The containers may, in some instances, contain saline, formalin, or other fixative solution. The kits may in some instances comprise an apparatus for performing a Pap smear. The kits may in some instances comprise an apparatus for obtaining an endometrial biopsy or sample. The kits may in some instances contain disposable sampling brushes which are configured to couple to a reusable handle, sometimes with both endocervical and ectocervical sampling brushes interchangeably couplable to the same handle. Advantages to providing components in kit form include, among others, decreased costs for packaging vs. individual packages, convenience, ease of maintaining sterility and avoiding contamination, and promoting routine sampling of the endocervical canal on sampling lesions of the ectocervix.

### Additional Embodiments

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Additionally, it is contemplated that various aspects and features of the invention described can be practiced separately, combined together, or substituted for one another, and that a variety of combination and sub-combinations of the features and aspects can be made and still fall within the scope of the invention. Further, the disclosure herein of any particular feature, aspect, method, property, characteristic, quality, attribute, element, or the like in connection with an embodiment can be used in all other embodiments set forth herein. Thus, it is intended that the scope of the present invention herein disclosed should not be limited by the particular disclosed embodiments described above, but should be determined only by a fair reading of the claims.

Conditional language, such as, among others, “may,” “could,” “might,” or “may,” unless specifically stated otherwise, or otherwise understood within the context as used, is generally intended to convey that certain embodiments include while other embodiments do not include, certain features or elements. Thus, such conditional language is not generally intended to imply that features or elements are in any way required for one or more embodiments.

What is claimed is:

1. An apparatus for obtaining a sample of cells in epithelial tissue of the body comprising:
a brush;
wherein said brush comprises bristles having sufficient stiffness to penetrate at least said two layers of said epithelial tissue;
wherein said brush is a non-lacerational brush; and
wherein the bristles collectively substantially form the shape of a cone.

2. An apparatus for obtaining a sample of cells in epithelial tissue of the body comprising:
a brush;
wherein said brush comprises bristles having sufficient stiffness to penetrate at least said two layers of said epithelial tissue;
wherein said brush is a non-lacerational brush; and
wherein the bristles collectively substantially form the shape of a cylinder.

3. An apparatus for obtaining a sample of cells in epithelial tissue of the body comprising:
a brush;
wherein said brush comprises bristles having sufficient stiffness to penetrate at least said two layers of said epithelial tissue;
wherein said brush is a non-lacerational brush; and
wherein the brush is shaped so that it is capable of collecting histological tissue samples from an endocervical canal.

4. The apparatus according to either of claims 1 or 2, wherein the brush is non-invasive upon insertion to the endocervical canal.

5. The apparatus according to claim 4, wherein the non-invasive brush comprising a sheath for shielding the brush from penetrating tissue upon entry to the endocervical canal.

6. The apparatus according to claim 4, wherein the size and/or stiffness of the bristles are sufficiently non-invasive.

7. The apparatus according to claim 3,
wherein the brush further comprises a twisted wire,
wherein bristles are held in place by the twisted wire and arranged from smallest in length at the distal end of the brush to the longest bristles at the proximal end of the brush to form a spiral substantially in the shape of a cone.

8. The apparatus according to claim 3,
wherein the brush further comprises a twisted wire,
wherein bristles are of uniform length and held in place by the twisted wire to form a spiral substantially in the shape of a cylinder.

9. The apparatus according to claim 8, wherein the brush further comprises a rounded tip or a cone-shaped tip on the distal end of the brush.

10. The apparatus according to claim 8, wherein said bristles are configured to collect cells from three layers of said epithelial tissue, said three layers comprising superficial, intermediate and basal layers, said basal layer separated from the submucosa by a basement membrane.

11. The apparatus according to claim 8, wherein said bristles have sufficient stiffness to penetrate said basement membrane and reach said submucosa.

12. The apparatus according to claim 3, further comprising
a handle,
wherein said handle comprises a distal and a proximal end;
wherein said brush is connected to said distal end; and
wherein the length of the twisted wire is positioned axially to the brush handle.

13. The apparatus according to claim 12, wherein said handle comprises a cylinder.

14. The apparatus according to claim 12 wherein said bristles directed outwardly from said twisted wire.

15. The apparatus according to claim 3, wherein said bristles have a tip stiffness, and wherein the tip stiffness of each bristle is between 0.04 and 0.2 lbs/inch.

16. The apparatus according to claim 15, wherein said bristles have a tip stiffness, and wherein said bristles protrude approximately 0.075 to 0.225 inches from the twisted wire.

17. The apparatus of claim 3 wherein a number of bristles per inch of brush head length is between 400-1400.

18. The apparatus of claim 3 wherein the TPL ratio is no less than 0.02.

19. The apparatus of claim 3 wherein the TPL ratio is no greater than 0.1.

20. A method to collect cells in epithelial tissue of the body comprising:
inserting the apparatus of claim 2 into a body cavity, wherein the body cavity contacts the bristles upon insertion, and
collecting cells from at least two layers of said epithelial tissue.

21. The method of claim 20, wherein said bristles collect cells from three layers of said epithelial tissue, said three layers comprising superficial, intermediate and basal layers.

22. The method of claim 21,
wherein a basement membrane is located below said basal layer; and
wherein said bristles penetrate said basement membrane.

23. The method of claim 20, wherein said brush is rotated and collects tissue samples along the length of the brush.

24. The method of claim 21, wherein the body cavity is the endocervical canal and tissue samples are collected from the endocervical canal and the transformation zone.

25. The method of claim 22, further comprising exerting sufficient pressure on a scrubbing surface in contact with said epithelial tissue to dislodge cells.

26. The method of claim 21 wherein exfoliated and loose cells are removed from the ectocervical region prior to insering the apparatus of claim 3 into the endocervix.

27. A disposable kit for diagnostic sampling of the cervix, said kit comprising:
a brush for penetrating an at least two layers of an epithelial tissue of an endocervical canal wherein said brush comprises bristles having sufficient stiffness to penetrate at least said two layers of said endocervical canal wherein said brush is a non-lacerational brush and wherein the bristles collectively substantially form the shape of a cone or cylinder; and
an at least one apparatus for penetrating at least two layers of an epithelial tissue of an ectocervix.

28. The disposable kit of claim 27, further comprising an at least one sterile container for holding a sample obtained by the brush.

29. The disposable kit of claim 27, further comprising an at least one swab suitable for performing a Pap smear.

30. The disposable kit of claim 27, further comprising an at least one apparatus suitable for taking an endometrial biopsy of a uterus.
